(19) United States
(12) Patent Application Publication
(10) Pub. No.: US 2024/0374780 A1
(43) Pub. Date: Nov. 14, 2024

(54) ANTIMICROBIAL COMPONENT FOR A WOUND DRESSING

(71) Applicant: Medtrade Products Limited, Cheshire (GB)

(72) Inventors: Jennifer Haynes, Cheshire (GB); Neil Johnson, Cheshire (GB)

(21) Appl. No.: 18/564,143

(22) PCT Filed: May 27, 2022
(86) PCT No.: PCT/GB2022/051373

§ 371 Date: Nov. 27, 2023

(30) Foreign Application Priority Data
May 28, 2021 (GB) 2107726.8

(51) Int. Cl. A61L 15/44; A61F 13/02; A61L 15/22; A61L 15/26; A61L 15/42; B32B 5/02; B32B 5/18; B32B 5/24; B32B 5/32; B32B 7/05; B32B 7/12

(52) U.S. Cl. CPC A61L 15/44; A61F 13/0289; A61L 15/225; A61L 15/26; A61L 15/425; B32B 5/022; B32B 5/18; B32B 5/245; B32B 5/32; B32B 7/05; B32B 7/12; A61L 2300/104; A61L 2300/208; A61L 2300/404; B32B 2260/021; B32B 2262/02; B32B 2266/0278; B32B 2535/00

(57) ABSTRACT

The present invention concerns an antimicrobial component for a wound dressing and wound dressing comprising the same. The antimicrobial component comprises a source of silver ions, a quaternary ammonium salt and an organic acid. The antimicrobial component demonstrates improved antimicrobial efficacy across a broad spectrum of microorganisms and can be utilised in wound dressings where increased antimicrobial efficacy and a broad spectrum of efficacy is required across multiple microorganisms. This will assist clinicians when treating infected chronic wounds or preventing surgical site infections.

### TECHNICAL FIELD OF THE INVENTION

The present invention relates to an antimicrobial component for a wound dressing and wound dressings comprising the same.

### BACKGROUND TO THE INVENTION

The antimicrobial effects of silver are widely known. Silver is effective at reducing infection while subjecting human cells to minimal toxicity. Silver's antimicrobial method of action is thought to include inhibition of oxidative enzymes and denaturation of membrane proteins, leading to damage of membrane function.

Silver containing dressings are used to treat infected chronic wounds and surgical site infections, typically in combination with systemic antibiotics. The role of the silver dressing is to absorb the infected exudate and kill bioburden within the dressing, while oral or intravenous antibiotics target the systemic infection.

Although there are many silver dressings already on the market which claim antimicrobial performance, the criteria set by the FDA for new antimicrobial products has recently changed. The FDA requirement to achieve 510 (k) clearance as an antimicrobial now dictates that the wound dressing demonstrates a log 4 reduction against three gram-positive bacteria, three gram-negative bacteria, a yeast and a mould when tested in a modified American Association of Textile Chemists (AATCC)-100 in-vitro test method.

A key difference of the modified AATCC-100 test method over previous antimicrobial performance tests required by the FDA is the inclusion of a preconditioning phase. In this phase, the dressings are exposed to significant quantities of simulated wound fluid sufficient to ‘spoil’ the dressing and represent clinical worst case, prior to inoculation with pathogenic microorganisms. The principle behind this pre-conditioning phase is that in a saturated dressing there is a risk that the antimicrobial components (such as silver ions) may interact with the components of the exudate (such as nutrients, proteins, growth factors, white blood cells and matrix metalloproteinases (MMPs)) and thus may be depleted or ‘less available’ to act upon microorganisms. A potential consequence of this would be a decrease in the antimicrobial efficacy of the product. As such, simulated wound fluid is used to saturate the dressings for a duration representative of the dressing wear time before the dressing is inoculated with microorganisms. This ensures the dressing is heavily spoiled and sufficiently challenged during the test method.

In addition, the inclusion of a yeast and a mould in the modified AATCC-100 test method presents another challenge, as the dressing must achieve a broad spectrum kill against these lesser researched organisms as well as multiple bacteria species.

The challenge of the preconditioning aspect of the modified AATCC-100 test method, in combination with the requirement for broad spectrum performance, make a log 4 reduction difficult to attain. Silver salts alone do not seem to be effective against the full spectrum of microorganisms, so inclusion of other actives is necessary to enhance their performance.

The present invention therefore aims to solve the above-mentioned problems by providing an antimicrobial component for a wound dressing that demonstrates improved antimicrobial efficacy across a broad spectrum of microorganisms and meets the aforementioned criteria for FDA approval as an antimicrobial.

### SUMMARY OF THE INVENTION

According to a first aspect of the present invention, there is provided an antimicrobial component for a wound dressing comprising a source of silver ions, a quaternary ammonium salt and an organic acid.

It has been surprisingly discovered that the combination of a source of silver ions, a quaternary ammonium salt and an organic acid provides an antimicrobial component with improved antimicrobial efficiency across a broad spectrum of microorganisms.

Surprisingly, the antimicrobial component according to the present invention demonstrates a greater than log 4 reduction (relative to a negative control) against three gram-positive bacterial species, three gram-negative bacterial species, a yeast and a mould when tested in an AATCC-100 modified test method.

The combination of a source of silver ions, a quaternary ammonium salt and an organic acid has a synergistic effect and beneficially produces an antimicrobial effect greater than the individual substances alone.

The inventors surprisingly discovered that the addition of a quaternary ammonium salt and an organic acid to a source of silver ions enhances the antimicrobial effectiveness of the source of silver ions when compared to the effectiveness of the source of silver ions alone. Moreover, the inventors surprisingly discovered that the addition of a source of silver ions and an organic acid to a quaternary ammonium salt enhances the antimicrobial effectiveness of the quaternary ammonium salt when compared to the effectiveness of the quaternary ammonium salt alone.

In short, the inventors surprisingly discovered that the combination of a source of silver ions, a quaternary ammonium salt and an organic acid provides an antimicrobial effect greater than the sum of the antimicrobial effects of the individual substances alone.

Without being bound by theory, it is hypothesised that the organic acid adjusts the pH of the environment within the antimicrobial component on absorption of aqueous fluids (such as wound exudate), thus enhancing silver ion's antimicrobial efficacy. Most chronic wounds have alkaline pH wound environments, while a shift to acidic pH is preferable for wound healing. In addition, many microorganisms struggle to survive at lower pH, due to reduced gene expression, enzyme activity and loss of membrane pH gradient.

Many bacteria and yeast species commonly associated with wound infection tend to thrive in neutral to alkaline conditions. When microorganisms are in environments out of their optimum growth pH they undergo ‘pH stress’, ultimately leading to intracellular acidification. Therefore, including an organic acid to reduce the pH of the exudate, creates an inhospitable environment for microorganisms, thus yielding superior performance relative to use of a source of silver ions and a quaternary ammonium salt either alone or in combination.

The mould included in the modified AATCC-100 test method, Aspergillus Brasiliensis, is not substantially affected by acidic pH. Aspergillus mould species are known to maintain stable growth between pH 2-11, a much larger range than the other microorganisms studied in the AATCC-100 test method. Consequently, the inclusion of a source of silver ions and organic acid is not sufficient to achieve a greater than log 4 reduction against Aspergillus Brasiliensis. However, through research it was found that the addition of a quaternary ammonium salt further boosted performance against this specific species, whilst also surprisingly enhancing the antimicrobial effectiveness of the component against the other microorganisms.

The antimicrobial component of the present invention may be incorporated in a wound dressing.

Therefore, in a further aspect of the present invention, there is provided a wound dressing comprising a source of silver ions, a quaternary ammonium salt and an organic acid.

Wound dressings of the present invention can be utilised in situations where increased antimicrobial efficacy and a broad spectrum of efficacy is required across multiple microorganisms. This will assist clinicians when treating infected chronic wounds or preventing surgical site infections, and the antimicrobial performance will not be compromised by the amount of exudate produced by the wound. This ability to maintain antimicrobial efficacy over the course of the full dressing wear time allows the clinician confidence to leave the dressing in place for longer without concern that the infection is not appropriately managed. Moreover, this reduces the burden on clinicians' time as a result of fewer dressing changes. Ultimately, this yields economic savings for healthcare providers and yields a lower environmental burden through a reduction in disposable clinical waste.

The term ‘wound’ is used herein to refer to any breach or opening in the skin or subcutaneous tissue at a physiological target site of a human or animal. Typically, the wound dressing of the present invention is applicable to a physiological target site of a human. The term physiological target site may also be referred to herein as a wound site. The term ‘infected wound’ refers to a wound which has an increased bioburden contributing to stalled healing. Signs and symptoms of infected wounds include pain, redness, heat, swelling and malodour. The dressing is suitable for application to infected wounds and non-infected wounds. These may include acute wounds, chronic wounds such as pressure ulcers and diabetic foot ulcers, and post-surgical wounds.

The term ‘antimicrobial’ generally refers to substances or components that can kill, or inhibit the growth of, microorganisms.

However, the modified AATCC-100 test method increases the criteria for a wound dressing to be considered antimicrobial. Instead, the AATCC-100 test method requires that in order for a wound dressing to claim antimicrobial efficacy it must demonstrate a log 4 bacterial kill rate within 24 hours.

Accordingly, the term ‘antimicrobial’ is used herein to refer to a substance or component that demonstrates a greater than log 4 bacterial kill rate within 24 hours.

There may be more than one source of silver ions. For example, there may be two, three, four or five sources of silver ions. In one embodiment, there may be a plurality of sources of silver ions.

The silver ions may be monovalent (Ag+), divalent (Ag2+), and/or trivalent (Ag3+).

The source of silver ions may be a source of ionic silver.

By the term “source of ionic silver” it is meant any substance which may provide one or more silver ions.

The source of silver ions may be a silver salt.

The silver salt may be selected from the group consisting of silver sulphate, silver citrate, silver acetate, silver carbonate, silver lactate, silver phosphate, silver chloride and/or combinations of any two or more thereof.

Preferably, the silver salt is silver chloride.

The source of silver ions may be present in an amount of from 2-5% by weight of the antimicrobial component. More preferably, the source of silver ions may be present in an amount of from 3-5% by weight of the antimicrobial component, most preferably the source of silver ions may be present in the antimicrobial component in an amount of 4% by weight of the antimicrobial component.

The source of silver ions may be present in an amount of from 50-89% of the combined weight of the source of silver ions, quaternary ammonium salt and organic acid. More preferably, the source of silver ions may be present in an amount of from 71-77% of the combined weight of the source of silver ions, quaternary ammonium salt and organic acid, most preferably the source of silver ions may be present in the antimicrobial component in an amount of 73% of the combined weight of the source of silver ions, quaternary ammonium salt and organic acid.

The source of silver ions may be present in an amount to yield at least 0.95, 1.00, 1.05, 1.10. 1.15 or at least 1.20 mg/cm2 of silver ions. The source of silver ions may be present in an amount to yield no more than 1.45, 1.40, 1.35, 1.30 1.25, 1.20, 1.15, or no more than 1.10 mg/cm2 of silver ions

The source of silver ions may be present in an amount to yield 0.95-1.45, 0.95-1.40, 0.95-1.35, 0.95-1.30, 0.95-1.25, 0.95-1.20, 1.00-1.45, 1.05-1.45, 1.10-1.45, 1.15-1.45, 1.20-1.45, 0.95-1.44 mg/cm2 of silver ions. More preferably, the source of silver ions may be present in an amount to yield 1.05-1.34 mg/cm2 of silver ions, most preferably the source of silver ions may be present in the antimicrobial component in an amount to yield 1.15 -1.25, 1.16-1.24, 1.17-1.23, 1.18 1.22, 1.19-1.21 mg/cm2 of silver ions. Most preferably still, the source of silver ions may be present in an amount to yield 1.20 mg/cm2 of silver ions.

The quaternary ammonium salt may be a non-animal derived quaternary ammonium salt.

By “non-animal derived” it is meant that the quaternary ammonium salt is not derived from the body of an animal. Beneficially, this minimises the risk of contamination of the dressing with animal pathogens,

The quaternary ammonium salt may be selected from the group consisting of benzalkonium chloride, benzethonium chloride and cetyltrimethylammonium chloride. Preferably, the quaternary ammonium salt is benzalkonium chloride.

The quaternary ammonium salt may be present in an amount of from 0.1-0.5% by weight of the antimicrobial component. Preferably, the quaternary ammonium salt is present in an amount of from 0.2-0.5% by weight of the antimicrobial component, more preferably 0.5% by weight of the antimicrobial component.

The quaternary ammonium salt may be present in an amount of from 1-16% of the combined weight of the source of silver ions, quaternary ammonium salt and organic acid. Preferably, the quaternary ammonium salt is present in an amount of from 3-12% of the combined weight of the source of silver ions, quaternary ammonium salt and organic acid, more preferably 9% of the combined weight of the source of silver ions, quaternary ammonium salt and organic acid.

The quaternary ammonium salt may be present in an amount of at least 0.16, 0.17, 0.18, 0.19, or at least 0.20 mg/cm2. The quaternary ammonium salt may be present in an amount of no more than 0.24, 0.23, 0.22, 0.21, or no more than 0.20 mg/cm2.

The quaternary ammonium salt may be present in an amount of from 0.15-0.24, 0.15-0.23, 0.15-0.22, 0.15-0.21, 0.15-0.20, 0.15-0.19, 0.15-0.24, 0.16-0.24, 0.17-0.24, 0.18-0.24, 0.16-0.24, 0.16-0.23, 0.16-0.22, 0.16-0.21, 0.16-0.20, 0.16-0.19, 0.17-0.23, or from 0.18-0.23 mg/cm2. Preferably, the quaternary ammonium salt is present in an amount of from 0.16-0.24 mg/cm2. More preferably, the quaternary ammonium salt is present in an amount of from 0.18-0.22 mg/cm2, more preferably still 0.19-0.21 mg/cm2, most preferably 0.20 mg/cm2.

The organic acid may be selected from the group consisting of lactic acid, citric acid, acetic acid, malic acid, glycolic acid and/or combinations of any two or more thereof. Preferably, the organic acid is lactic acid.

The organic acid may be present in an amount of from 0.5-1.5% by weight of the antimicrobial component. Preferably, the organic acid is present in an amount of from 0.75-1.25% by weight of the antimicrobial component. More preferably, the organic acid may be present in an amount of 1% by weight of the antimicrobial.

The organic acid may be present in an amount of from 8-40% of the combined weight of the source of silver ions, quaternary ammonium salt and organic acid. Preferably, the organic acid is present in an amount of from 18-22% of the combined weight of the source of silver ions, quaternary ammonium salt and organic acid. More preferably, the organic acid may be present in an amount of 18% of the combined weight of the source of silver ions, quaternary ammonium salt and organic acid.

The organic acid may be present in an amount of at least 0.26, 0.27, 0.28, 0.29, or at least 0.30 mg/cm2. The organic acid may be present in an amount of no more than 0.38, 0.37, 0.36, 0.35, 0.34, 0.33, 0.32, 0.31, or no more than 0.30 mg/cm2.

The organic acid may be present in an amount of from 0.26-0.38, 0.26-0.37, 0.26-0.35, 0.27-0.35, 0.28-0.34, 0.29-0.34, 0.30-0.34, 0.30-0.33, 0.30-0.32, 0.31-0.34, or from 0.32-0.34 mg/cm2. Preferably, the organic acid is present in an amount of from 0.30-0.34 mg/cm2. More preferably, the organic acid is present in an amount of from 0.31-0.33 mg/cm2, more preferably still 0.32 mg/cm2.

The pH of the antimicrobial component may be at least 1, at least 2 or at least 3. The pH of the antimicrobial component may be no more than 5, 4 or no more than 3.5.

The pH of the antimicrobial component may be from 1-6, 2-6, 2-5, 2-4, 3-4 or from 3-3.5.

Preferably, the pH of the antimicrobial component is between 3-5. More preferably, the pH is from 3-3.5. Beneficially, this improves the antimicrobial effectiveness of the antimicrobial component by inducing ‘pH stress’ in the microorganisms as described herein.

The source of silver ions, quaternary ammonium salt and organic acid may be present in a weight ratio of at least 2:0.1:0.5, 2.5:0.2:0.75 or at least 3.0:0.3:1.

The source of silver ions, quaternary ammonium salt and organic acid may be present in a weight ratio of no more than 5:0.5:1.5, 4:0.4:1.25, or no more than 3.5:0.3:1.

The source of silver ions, quaternary ammonium salt and organic acid may be present in a weight ratio of from 2:0.1:0.5 to 5:0.5:1.5, or 3:0.2:0.75 to 5:0.5:1.25.

Preferably, the antimicrobial component comprises a source of silver ions in an amount of from 2-5% by weight of the antimicrobial component, a quaternary ammonium salt in an amount of from 0.1-0.5% by weight of the antimicrobial component and an organic acid in an amount of from 0.5-1.5% by weight of the antimicrobial component.

Preferably, the antimicrobial component comprises a source of silver ions in an amount to yield 1.05-1.34 mg/cm2 of silver ions, a quaternary ammonium salt in an amount of from 0.18-0.22 mg/cm2 and an organic acid in an amount of from 0.30-0.34 mg/cm2

The antimicrobial component of the present invention may further comprise an absorbent material.

In a further aspect of the present invention, there is provided an absorbent material for a wound dressing comprising a source of silver ions, a quaternary ammonium salt and an organic acid

The term ‘absorbent material’ is used herein to refer to a physiologically acceptable material that is capable of absorbing liquid, such as wound exudate, and which is capable of absorbing liquid to greater than about 500% by weight of the absorbent material, and with a liquid retention of greater than about 40%.

The absorbent material may comprise a fibrous, foam, non-woven or woven material.

Preferably, the absorbent material is a polyurethane foam. The polyurethane foam may be an open cell foam. The polyurethane foam may be hydrophilic. The polyurethane foam may have a thickness of from 2-5 mm. Preferably the polyurethane foam is present at a thickness of 3.0 mm.

In one embodiment, the absorbent material may comprise more than one absorbent material.

In such an embodiment, the absorbent material may comprise a polyurethane foam and a non-woven material.

The non-woven material may be in the form of fibres. Typically, the non-woven material is in the form of non-woven fibres. The length of the fibres can be up to about 100 mm, and is typically from about 20-75 mm, more typically from about 32 to 51 mm.

The non-woven material may be selected from the group consisting of polyester. viscose or polyolefin fibres, or combinations thereof. The non-woven material may comprise or consist of an air laid nonwoven fibre. By air laid nonwoven fibre, it is meant a continuous web formed by a mixture of short fibres and/or 100% pulped fibres.

The absorbent material may comprise a chemical pulp made from long fibre soft woods. Preferably, the absorbent material may be a fluff pulp. By the term fluff pulp, it is meant a chemical pulp made of cellulose fibres obtained from long softwoods.

The absorbent material may comprise, or consist of, a gelling or semi-gelling material.

The term ‘gelling material’ is used herein to refer to a material in which substantially all of the components therein may gel upon contact with water or body fluid(s). For example, it may comprise a fibrous material wherein substantially all of the fibres are capable of gelling upon contact with water or body fluid(s).

The term ‘semi-gelling’ is used herein to refer to a material that comprises a mixture of components, some of which gel upon contact with water or body fluid(s) and some of which do not. For example, a semi-gelling absorbent material may comprise a combination of fibres, some of which gel upon contact with water or body fluid(s) and some of which do not.

The gelling or semi-gelling material may be in any available form, such as for example, fibres, granules, powder, flakes, sheet, foam, freeze dried foam, compressed foam, film, perforated film, beads, and combinations of two or more of the aforesaid.

The gelling or semi-gelling material may be selected from carboxymethylcellulose, alginate, chitosan salt or a chitosan salt derivative.

Typically, the gelling or semi gelling material is in the form of fibres. The fibres can be of any desired diameter or length and can be formed into a textile fabric or a pad for use. The fibres may be woven or non-woven. Preferably, the fibres are non-woven.

The absorbent material may be discontinuous. In such an embodiment, the absorbent material may comprise one or more fenestrations. The fenestrations may comprise slits, openings or perforations. The fenestrations may penetrate through the entire thickness of the absorbent material. Alternatively, the fenestrations may penetrate partially through the thickness of the absorbent material. Preferably, the fenestrations extend through the entire thickness of the absorbent material.

The fenestrations may be arranged in one or more directions. The fenestrations may be arranged in a linear repeating pattern. Alternatively, the fenestrations may be arranged in a random configuration.

The fenestrations facilitate the flow of wound exudate through the antimicrobial component and improve the conformability of the antimicrobial component when incorporated into a wound dressing.

Alternatively, the absorbent material may be continuous. In such an embodiment, the absorbent material may comprise no fenestrations. Beneficially, when incorporated in a wound dressing, a non-fenestrated absorbent material in the antimicrobial component may prevent the passage of any gelling absorbent material present in the wound dressing, from passing through the antimicrobial component and into the wound.

The source of silver ions, organic acid, and quaternary ammonium salt may be distributed throughout the absorbent material.

Preferably, the source of silver ions, organic acid, and quaternary ammonium salt are uniformly distributed throughout the absorbent material.

The antimicrobial component of the present invention may have a laminate structure. The laminate structure may comprise two or more layers. The laminate structure may comprise two, three, four, five, six, seven, eight, nine, ten or more layers.

It has been observed that the provision of an antimicrobial component having a laminate structure demonstrates improved dressing flexibility and extensibility. Beneficially, this improves conformability of the subsequent wound dressing.

The layers of the laminate structure may be secured together by an adhesive.

The adhesive may be a powder adhesive.

The powder adhesive may be a heat activated adhesive.

The powder adhesive may comprise polycaprolactone.

Preferably, the powder adhesive is a heat activated polycaprolactone powder adhesive.

Preferably, the layers of the laminate structure are not secured together by an acrylic adhesive.

Surprisingly, the inventors discovered that the presence of an acrylic adhesive in the dressing reduced the antimicrobial effectiveness of the composition.

The laminate structure may comprise one or more absorbent materials.

In an embodiment wherein the antimicrobial component comprises a laminate structure of two layers, the silver ions, the organic acid and the quaternary ammonium salt may be distributed throughout a first layer and the first layer may be secured to a second layer.

Preferably, in an embodiment wherein the antimicrobial component comprises a laminate structure of two layers, the silver ions, the organic acid and the quaternary ammonium salt may be uniformly distributed throughout a first layer and the first layer may be secured to a second layer.

Preferably, the laminate structure may comprise a first layer of polyurethane foam and a second layer of non-woven material.

Beneficially, a laminate structure comprising a first layer of polyurethane foam and a second layer of non-woven material improves the structural integrity of the dressing. For example, the non-woven second layer prevents the foam first layer from expanding and distorting when saturated.

In such an embodiment, the first layer may comprise the source of silver ions, the organic acid and the quaternary ammonium salt.

The pH of absorbent material may be at least 2 or at least 3. The pH of the absorbent material may be no more than 5, 4 or 3.

Preferably, the pH of the absorbent material may be from 2-4.

The laminate structure may comprise a first layer and a second layer, wherein the first layer comprises a source of silver ions and an organic acid, and wherein the second layer comprises a quaternary ammonium salt.

The silver ions and the organic acid may be uniformly distributed throughout the first layer and the quaternary ammonium salt may be uniformly distributed throughout the second layer.

Preferably, the laminate structure may comprise a first layer of polyurethane foam and a second layer of polyurethane foam, wherein the first layer comprises the source of silver ions and the organic acid, and wherein the second layer comprises the quaternary ammonium salt.

Surprisingly, an antimicrobial component having a laminate comprising a first layer of polyurethane foam and a second layer of polyurethane foam, wherein the first layer comprises the source of silver ions and the organic acid, and wherein the second layer comprises the quaternary ammonium has improved antimicrobial efficacy. Without wishing to be bound by theory, it is thought that the source of silver ions is more reliant on an acidic pH environment to improve its antimicrobial activity than the quaternary ammonium salt. In this embodiment, the source of silver ions is initially exposed to a greater content of organic acid when compared to an antimicrobial component having a source of silver ions, organic acid and quaternary ammonium salt present together in a single layer. In this regard, it thought that the antimicrobial effect of the source of silver ions is improved to a greater extent by the presence of an organic acid in comparison to the improvement of the antimicrobial effect of the quaternary ammonium salt when in the presence of an organic acid.

The antimicrobial component may be a part of a wound dressing.

Therefore, in a further aspect of the present invention, there is provided a wound dressing comprising an antimicrobial component for a wound dressing comprising a source of silver ions, a quaternary ammonium salt and an organic acid.

The wound dressing is intended to be applied to any wound which would benefit from a wound dressing having antimicrobial performance, in particular infected chronic wounds or post-surgical wounds. As such, it is not limited to a particular size or shape. Typically, the wound dressing has a generally rectangular shape and preferably a square shape.

The wound dressing may be placed in direct or indirect contact with the wound.

The wound dressing may have a wound contact layer. The wound contact layer may directly contact the wound.

The wound contact layer may have a laminate structure. The laminate structure may comprise two or more layers. The laminate structure may comprise two, three, four, five, six, seven, eight, nine, ten or more layers.

Preferably, the wound contact layer comprises a bilaminate structure. The bilaminate structure may have a distal carrier layer and a proximal layer. The proximal layer may comprise an adhesive for contacting and securing the wound contact layer to the wound and/or the skin surrounding the wound. The distal layer may comprise a film that the adhesive layer is coated onto.

The terms ‘proximal’ and ‘distal’ are used relative to a wound site. For example, the term ‘proximal surface’ is used herein to refer to a surface of a component of the dressing that, in use, faces toward the wound site and the term ‘distal surface’ is used herein to refer to a surface of a component of the dressing that, in use, faces away from the wound site. Similarly, a ‘proximal layer’ can be used to refer to a layer that is located closer to the wound and a ‘distal layer’ can be used to refer to a layer that is located away from the wound.

The wound contact layer may comprise one or more perforations to facilitate the passage of wound exudate to the interior of the dressing. The wound contact layer may comprise a plurality of perforations.

The perforations may be arranged in one or more directions on the wound contact layer. The perforations may be arranged in a linear repeating pattern. Alternatively, the perforations may be arranged in a random configuration.

The perforations may extend fully through the wound contact layer. Alternatively, the perforations extend partially through the wound contact layer.

The perforations may be substantially circular. The perforations may have a diameter of from 0.5-10 mm.

The perforations in the wound contact layer increases the rate of uptake of wound exudate from the wound contact layer, thereby improving the effectiveness of the wound dressing. The perforations in the wound contact layer also reduce the adhesion of the wound contact layer to the wound and/or the skin surrounding the wound. Advantageously, this reduces damage to the wound area upon removal of the dressing.

The proximal layer of the wound contact bilaminate preferably comprises a biocompatible pressure sensitive adhesive. The adhesive may be any suitable skin-contact adhesive known in the art. The adhesive may comprise a silicone adhesive or polyurethane adhesive. Typically, the adhesive is a silicone adhesive, such as polydimethylsiloxane. Alternatively, the adhesive can be a solvent or water based acrylic adhesive, a polyurethane adhesive, a hydrogel adhesive such as for example an atraumatic hydrogel adhesive, or any combinations thereof. The adhesive is intended to create a tight seal between the wound dressing and the patient's skin surrounding the wound.

The proximal layer of adhesive may be coated onto the proximal surface of the carrier layer with a coat weight of from 30 to 300 gsm.

The carrier layer of the wound contact bilaminate may comprise, or consist of, a polyurethane film. Alternatively, the carrier layer of the wound contact bilaminate may comprise, or consist of, a polyethylene film. Preferably, the carrier layer comprises polyurethane film

The wound contact layer is not limited to a particular size or shape unless specifically defined herein. The wound contact layer may take a variety of sizes or shapes as desired or as appropriate. Typically, the wound contact layer has a generally rectangular shape. Preferably, the wound contact layer has a generally square shape.

The wound dressing may have a backing layer.

The backing layer may serve as a barrier and may be operable to prevent microorganisms, such as bacteria, from entering the wound dressing from an external source, such as clothing, etc. Further, the backing layer is also operable to retain wound exudate within the wound dressing and prevent it leaching out of the wound dressing.

The backing layer may be gas-permeable. The backing layer may be substantially impermeable to microorganisms, such as bacteria. The backing layer may be substantially impermeable to liquids.

The permeability of the backing layer to gases, such as air and moisture vapour, permits the transmission of moisture vapour through its structure. This facilitates the transpiration of wound exudate from the dressing into the external environment. Beneficially, this increases the breathability of the dressing and prevents the saturation of the dressing with exudate, which leads to a reduced number of dressing changes.

The backing layer may have a moisture vapour transmission rate in the range of 50 g/100 cm2/24 hours to 250 g/100 cm2/24 hours, or from 50 g/100 cm2/24 hours to 250 g/100 cm2/24 hours. Good results are observed when the backing layer has a moisture vapour transmission rate in the range of 80 g/100 cm2/24 hours to 160 g/100 cm2/24 hours or from 100 g/100 cm2/24 hours to 250 g/100 cm2/24 hours. In a preferred embodiment, the backing layer has a moisture vapour transmission rate of 100 g/100 cm2/24 hours to 150 g/100 cm2/24 hours or from 100 g/100 cm2/24 hours to 250 g/100 cm2/24 hours.

The backing layer may comprise a material that is gas-permeable and liquid-impermeable. The backing layer may also comprise a material that is microorganism-impermeable.

The backing layer may comprise, or consist of, any biologically acceptable polymer material that is liquid-and/or microorganism-impermeable but gas-permeable. Suitable biologically acceptable polymer materials for the backing layer may be selected from the group consisting of polyurethane and polyethylene.

The backing layer may be in the form of a film, a foam, or a combination thereof. Preferably, the backing layer is in the form of a film.

Preferably, the backing layer comprises, or consists of, a polyurethane film.

The polyurethane film typically has a weight of 5-40 gsm. The backing layer may have a thickness of from 5-50 microns, preferably 10-30 microns.

The backing layer may have a surface area greater than that of the antimicrobial component.

The backing layer is not limited to a particular size or shape unless specifically defined herein. The backing layer may take a variety of sizes or shapes as desired or as appropriate. Typically, the backing layer has a generally rectangular shape. Preferably, the backing layer has a generally square shape.

The antimicrobial component may be located between the wound contact layer and the backing layer.

The antimicrobial component may be attached to the wound contact layer by welding. In such an embodiment, the antimicrobial component may be attached to the wound contact layer by ultrasonic, heat or radio frequency welding. Preferably, the antimicrobial component is attached to the wound contact layer by heat welding.

In such an embodiment, the antimicrobial component is attached to the wound contact layer at a periphery of the antimicrobial component.

Preferably, the antimicrobial component is not attached to the wound contact layer by an acrylic adhesive.

Surprisingly, the inventors found that attaching the antimicrobial component to the wound contact layer using an acrylic adhesive reduces the antimicrobial effectiveness of the composition.

The antimicrobial component may be attached to the backing layer by welding. In such an embodiment, the antimicrobial component may be attached to the backing layer by ultrasonic, heat or radio frequency welding. Preferably, the antimicrobial component is attached to the backing layer by heat welding.

Alternatively, the antimicrobial component may not be substantially attached or not attached to the backing layer.

In such an embodiment, the antimicrobial component may be lightly adhered to the backing layer.

Preferably, the antimicrobial component is not substantially attached to the backing layer. This beneficially provides a higher moisture vapour transmission rate due to the absence of adhesive securing the antimicrobial component to the backing layer.

Preferably, the antimicrobial component is not attached to the backing layer by an acrylic adhesive.

Surprisingly, the inventors discovered that that attaching the antimicrobial component to the backing layer using an acrylic adhesive reduces the antimicrobial effectiveness of the composition.

The wound contact layer may be attached to the backing layer by welding. In such an embodiment, the wound contact layer may be attached to the backing layer by ultrasonic, heat or radio frequency welding. Preferably, the wound contact layer is attached to the backing layer by heat welding.

In one embodiment, the wound contact layer may have a first border region and the backing layer may have a second border region.

In such an embodiment, a first border region of the wound contact layer may be attached to a second border region of the backing layer by welding. The first border region of the wound contact layer may be attached to the second border region of the backing layer by ultrasonic. heat or radio frequency welding. Preferably, the first border region of the wound contact layer is attached to the second border region of the backing layer by heat welding.

In such an embodiment, the wound contact layer and the backing layer are attached to each other by adhesion between the first border region and the second border region. The border regions may surround the antimicrobial component.

The backing layer may be attached to the wound contact layer in all or part of the border regions.

Preferably, the backing layer is not attached to the wound contact layer by an acrylic adhesive.

Surprisingly, the inventors discovered that attaching the wound contact layer to the backing layer using an acrylic adhesive reduces the antimicrobial effectiveness of the composition.

The wound dressing may further comprise a removable protecting layer. The removable protecting layer may be a peelable protecting layer. The removable protecting layer may cover the proximal surface of the wound contact layer. The protecting layer facilitates storage of the wound dressing without detriment to the skin-contact adhesive on the proximal surface of the wound contact layer or forming the proximal layer of the wound contact trilaminate. The protecting layer is intended for removal prior to application of the dressing to a wound.

The removable protecting layer may comprise one, two or more parts that are separately removable via one, two or more tabs.

The wound dressing may comprise one or more absorbent layers. The absorbent layers may be located on a distal surface of the antimicrobial component.

The one or more absorbent layers may comprise an absorbent material as described herein.

The antimicrobial component may form a core of the wound dressing.

According to a further aspect of the present invention there is provided a method of manufacturing an antimicrobial component as defined herein comprising the step of bringing together a source of silver ions, a quaternary ammonium salt and an organic acid.

The method may comprise the step of incorporating a source of silver ions, a quaternary ammonium salt and an organic acid into an absorbent material.

Typically, the source of silver ions, the quaternary ammonium salt and the organic acid are incorporated into the absorbent material during the formation of the absorbent material. The method may comprise the steps of:
a) incorporating a source of silver ions and an organic acid into a first absorbent material to form a first layer.
b) incorporating a quaternary ammonium salt into a second absorbent material to form a second layer; and
c) bringing together the first layer and the second layer.

The step of bringing together the first layer and the second layer may comprise adhering the first layer to the second layer using an adhesive, as described herein.

Preferably, in such an embodiment, the source of silver ions and the organic acid are uniformly distributed throughout the first layer and the quaternary ammonium salt is uniformly distributed throughout the second layer.

Alternatively, the method may comprise the steps of:
a) incorporating a source of silver ions, a quaternary ammonium salt and an organic acid into a first absorbent material to form a first layer.
b) bringing together the first layer with a second layer of absorbent material

Preferably, the source of silver ions, the quaternary ammonium salt and the organic acid are uniformly distributed throughout the first layer.

There is also provided a method of manufacturing a wound dressing comprising an antimicrobial component as defined herein.

Therefore, according to a further aspect of the present invention, there is provided a method of manufacturing a wound dressing comprising the steps of:
a) providing a wound contact layer and a backing layer; and
b) locating an antimicrobial component as defined herein between the wound contact layer and the backing layer.

The method may further comprise the step of welding the wound contact layer to the backing layer.

The step of welding the wound contact layer to the backing layer may comprise ultrasonic, heat and/or radio frequency welding. Preferably, the step of welding comprises heat welding.

In one embodiment, the method may comprise the step of welding a first border region of the wound contact layer to a second border region of the backing layer.

According to a further aspect of the present invention there is provided an antimicrobial component according to the present invention for use as a medicament.

According to a further aspect of the present invention, there is provided an antimicrobial component according to the present invention for use in killing or inhibiting the growth of microorganisms.

According to a further aspect of the invention, there is provided a wound dressing incorporating an antimicrobial component according to the present invention for use in absorbing fluid discharged from a physiological target, or for use in stemming a flow of a fluid discharged from a physiological target site.

The further aspects of the present invention may incorporate any of the features of the other aspects of the invention described herein as desired or as appropriate.

### DETAILED DESCRIPTION OF THE INVENTION

In order that the invention may be more clearly understood, one or more embodiments thereof will now be described, by way of example only, with reference to the accompanying drawings of which:

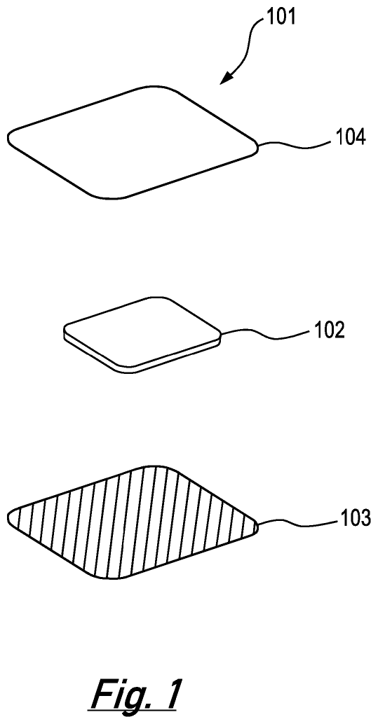

FIG. 1: shows an exploded view of a first embodiment of an antimicrobial component incorporated into a wound dressing.

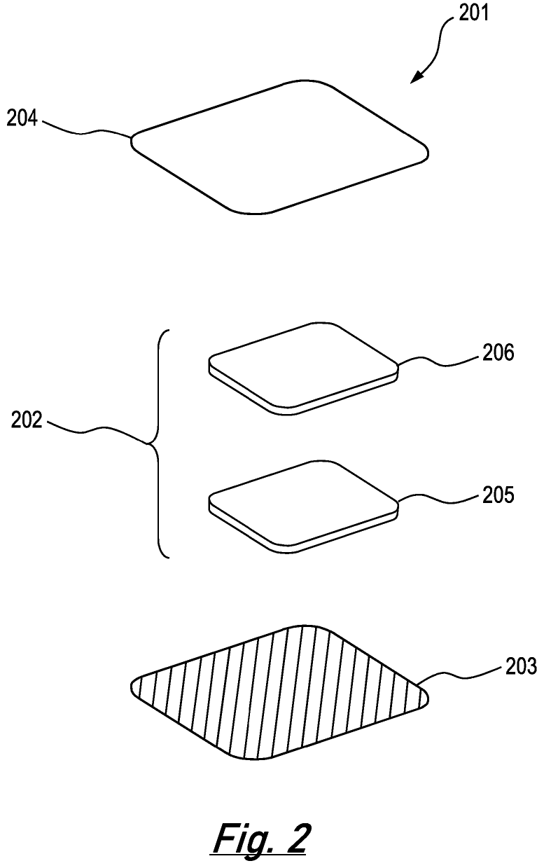

FIG. 2: shows an exploded view of a second embodiment of an antimicrobial component incorporated into a wound dressing.

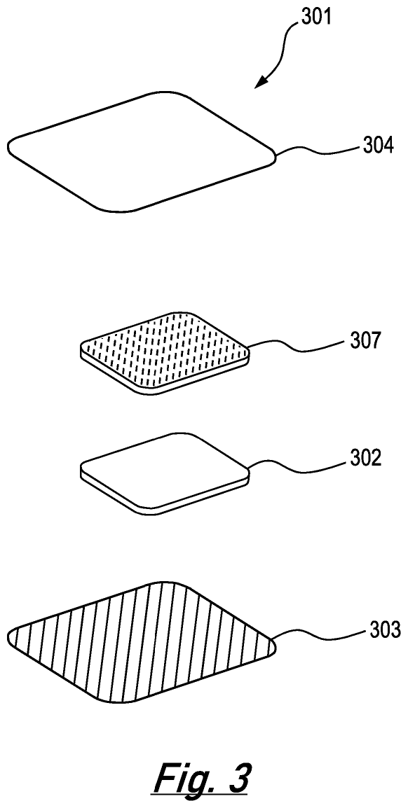

FIG. 3: shows an exploded view of a third embodiment of an antimicrobial component incorporated into a wound dressing.

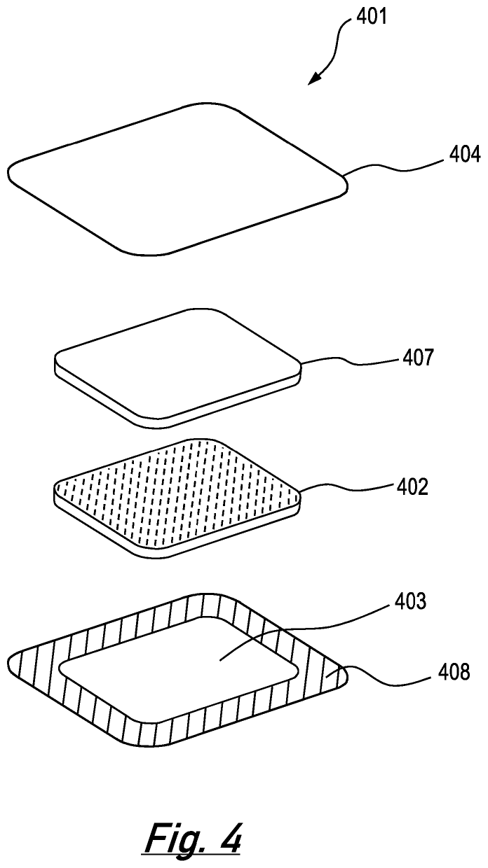

FIG. 4: shows an exploded view of a fourth embodiment of an antimicrobial component incorporated into a wound dressing.

Referring to FIG. 1, there is shown a multi-layered wound dressing (101) incorporating an antimicrobial component (102) according to the present invention. The antimicrobial component (102) comprises a polyurethane foam, silver chloride in an amount to yield 1.20 mg/cm2 of Ag+ ions or of 4% by weight of the antimicrobial component (102), benzalkonium chloride in an amount of 0.20 mg/cm2 or 0.5% by weight of the antimicrobial component (102), lactic acid in an amount of 0.32 mg/cm2 or 1% by weight of the antimicrobial component (102). The wound dressing also further comprises a silicone wound contact layer (103) and a polyurethane film backing layer (104).

Referring to FIG. 2, there is shown a multi-layered wound dressing (201) incorporating an antimicrobial component (202) according to the present invention. The antimicrobial component comprises a first layer (206) comprising a polyurethane foam, silver chloride in an amount to yield 1.20 mg/cm2 of Ag+ ions or of 4% by weight of the antimicrobial component (202) and lactic acid in an amount of 0.32 mg/cm2 or 1% by weight of the antimicrobial component. The antimicrobial component (202) also comprises a second layer (205) comprising a polyurethane foam and benzalkonium chloride in an amount of 0.20 mg/cm2 or 0.5% by weight of the antimicrobial component (202). The wound dressing also further comprises a silicone wound contact layer (203) and a polyurethane film backing layer (204).

Referring to FIG. 3, there is shown a multi-layered wound dressing (301) incorporating an antimicrobial component (302) according to the present invention. The antimicrobial component (302) comprises a polyurethane foam, silver chloride in an amount to yield 1.20 mg/cm2 of Ag+ ions or of 4% by weight of the antimicrobial component (302), benzalkonium chloride in an amount of 0.20 mg/cm2 or 0.5% by weight of the antimicrobial component (302) and lactic acid in an amount of 0.32 mg/cm2 or 1% by weight of the antimicrobial component (302). The wound dressing also further comprises a silicone wound contact layer (303), a polyurethane film backing layer (304) and an absorbent layer (307) comprising a non-woven material. The non-woven material is a polyester fibre.

Referring to FIG. 4, there is shown a multi-layered wound dressing (401) incorporating an antimicrobial component (402) according to the present invention. The antimicrobial component (402) comprises a carboxymethylcellulose non-woven fibre, silver chloride in an amount to yield 1.20 mg/cm2 of Ag+ ions or of 4% by weight of the antimicrobial component (402), benzalkonium chloride in an amount of 0.20 mg/cm2 or 0.5% by weight of the antimicrobial component (402), lactic acid in an amount of 0.32 mg/cm2 or 1% by weight of the antimicrobial component (402). The wound dressing also further comprises a silicone wound contact layer (403), a polyurethane film backing layer (404) and an absorbent layer (407) comprising an absorbent material. The absorbent material is a polyurethane foam. The silicone wound contact layer (403) has a first border region (408) for securing to a proximal surface of the backing layer (404).

In use, when a human or animal suffers a penetrating wound, the wound dressing (101, 201, 301, 401) is applied to the wound, with the wound contact layer (103, 203, 303, 403) being placed in direct contact with the wound. Upon the absorbance of wound exudate, the antimicrobial component is able to kill, or inhibit the growth of, microorganisms in the wound dressing.

### EXAMPLE ANTIMICROBIAL COMPONENTS

The following are examples of antimicrobial components.

### Example 1

An antimicrobial component comprising a first layer comprising a polyurethane foam, silver chloride in an amount to yield 0.95 mg/cm2 Ag+ ions or of 4% by weight of the antimicrobial component and lactic acid in an amount of 0.32 mg/cm2 or 1% by weight of the antimicrobial component, and a second layer comprising a polyurethane foam and benzalkonium chloride in an amount of 0.16 mg/cm2 or 0.5% by weight of the antimicrobial component. The first layer is secured to the second layer by a polycaprolactone powder adhesive.

### Example 2

An antimicrobial component comprising a first layer comprising a polyurethane foam, silver carbonate in an amount to yield 0.95 mg/cm2 Ag+ ions or of 4% by weight of the antimicrobial component and citric acid in an amount of 0.32 mg/cm2 or 1% by weight of the antimicrobial component, and a second layer comprising a polyurethane foam and benzalkonium chloride in an amount of 0.16 mg/cm2 or 0.5% by weight of the antimicrobial component. The first layer is secured to the second layer by a polycaprolactone powder adhesive.

### Example 3

An antimicrobial component comprising a first layer comprising a polyurethane foam, silver acetate Win an amount to yield 0.95 mg/cm2 Ag+ ions or of 4% by weight of the antimicrobial component and citric acid in an amount of 0.32 mg/cm2 or 1% by weight of the antimicrobial component and a benzalkonium chloride in an amount of 0.16 mg/cm2 or 0.5% by weight of the antimicrobial component, and a second layer comprising a non-woven material. The first layer is secured to the second layer by a polycaprolactone powder adhesive.

### Example 4

An antimicrobial component comprising a first layer comprising a polyurethane foam, silver chloride in an amount to yield 0.95 mg/cm2 Ag+ ions or of 4% by weight of the antimicrobial component, lactic acid in an amount of 0.32 mg/cm2 or 1% by weight of the antimicrobial component and benzethonium chloride in an amount of 0.16 mg/cm2 or 0.5% by weight of the antimicrobial component, and a second layer comprising a polyurethane foam. The first layer is secured to the second layer by a polycaprolactone powder adhesive.

### Example 5

An antimicrobial component comprising a first layer comprising a polyurethane foam, silver phosphate in an amount to yield 0.95 mg/cm2 Ag+ ions or of 4% by weight of the antimicrobial component and acetic acid in an amount of 0.32 mg/cm2 or 1% by weight of the antimicrobial component and a benzalkonium chloride in an amount of 0.16 mg/cm2 or 0.5% by weight of the antimicrobial component, and a second layer comprising a non-woven material. The first layer is secured to the second layer by a polycaprolactone powder adhesive.

### Example 6

An antimicrobial component comprising a first layer comprising a polyurethane foam, silver citrate in an amount to yield 0.95 mg/cm2 Ag+ ions or of 4% by weight of the antimicrobial component and malic acid in an amount of 0.32 mg/cm2 or 1% by weight of the antimicrobial component and a benzalkonium chloride in an amount of 0.16 mg/cm2 or 0.5% by weight of the antimicrobial component, and a second layer comprising a non-woven material. The first layer is secured to the second layer by a polycaprolactone powder adhesive.

### Example 7

An antimicrobial component comprising a first layer comprising a polyurethane foam, silver sulphate in an amount to yield 0.95 mg/cm2 Ag+ ions or of 4% by weight of the antimicrobial component and lactic acid in an amount of 0.32 mg/cm2 or 1% by weight of the antimicrobial component and a cetyltrimethylammonium chloride in an amount of 0.16 mg/cm2 or 0.5% by weight of the antimicrobial component, and a second layer comprising a non-woven material. The first layer is secured to the second layer by a polycaprolactone powder adhesive.

### Reference Example 8

An antimicrobial component comprising a polyurethane foam and silver chloride in an amount to yield 0.95 mg/cm2 Ag+ ions or of 4% by weight of the antimicrobial component.

### Reference Example 9:

An antimicrobial component comprising a polyurethane foam and benzalkonium chloride in an amount of 0.16 mg/cm2 or 0.5% by weight of the antimicrobial component.

### Reference Example 10

An antimicrobial component comprising a polyurethane foam and lactic acid in an amount of 0.32 mg/cm2 or 1% by weight of the antimicrobial component.

### Reference Example 11:

An antimicrobial component comprising a first layer comprising a polyurethane foam, silver chloride in an amount to yield 0.95 mg/cm2 Ag+ ions or of 4% by weight of the antimicrobial component and benzalkonium chloride in an amount of 0.064 mg/cm2 or 0.2% by weight of the antimicrobial component, and a second layer comprising a non-woven material.

### Reference Example 12

An antimicrobial component comprising a first layer comprising a polyurethane foam, silver chloride in an amount to yield 0.95 mg/cm2 Ag+ ions or of 4% by weight of the antimicrobial component and lactic acid in an amount of 0.32 mg/cm2 or 1% by weight of the antimicrobial component, and a second layer comprising a non-woven material.

### Reference Example 13

An antimicrobial component comprising a first layer comprising a polyurethane foam, lactic acid in an amount of 0.32 mg/cm2 or 1% by weight of the antimicrobial component, and a second layer comprising a polyurethane foam and benzalkonium chloride in an amount of 0.16 mg/cm2 or 0.5% by weight of the antimicrobial component. The first layer is secured to the second layer by a polycaprolactone powder adhesive.

### Test Data

Antimicrobial Efficacy Test Method (Modification of AATCC-100):

In order to determine the efficacy of the antimicrobial component against microorganisms, the above examples were exposed to three gram-negative bacteria, three gram-positive bacteria, one yeast and one mould in the modified AATCC-100 test method.

### Test Procedure:

a) The examples were aseptically prepared and cut to a size of 4.2×4.2 cm.
b) Simulated wound fluid (SWF) is then prepared aseptically as 50% Foetal Calf Serum (heat activated) and 50% peptone diluent.
c) The examples are then fully saturated with SWF and placed in a humidified environment at 37° C. for 6 days (preconditioning phase).
d) After preconditioning, the examples are removed from the SWF and placed in a clean sterile petri dish.
e) Bacterial suspensions are prepared at approximately 1×106 CFUml-1 in an appropriate growth media.
f) The inoculums are enumerated by performing 10-fold dilutions plating out the resulting suspensions onto an appropriate media.
g) One millilitre of bacterial suspension is used to inoculate the examples that have been pre-conditioned for 6 days and the examples are left to incubate for 24 hours.
h) Following the 24 hr incubation period, the examples are placed into an appropriate neutralizer and viable organisms are recovered by sonication.
i) Microorganisms are enumerated by performing 10-fold dilutions of the neutralizer in growth media and plating the resulting suspensions on to appropriate media.
j) The log10 reduction is then calculated. At least a 4 log10 reduction is needed for all tested microorganisms.

The results of this test are shown in Table 1.

TABLE 1
Modified AATCC-100 test method results (Acceptance Criteria >4.0)

| | Average reduction (Log₁₀ CFU/Sample) | | | | | | |
|---|---|---|---|---|---|---|---|
| | Reference | Reference | Reference | Reference | Reference | Reference | |
| Microorganisms | Example 8 | Example 9 | Example 10 | Example 11 | Example 12 | Example 13 | Example 1 |
| Staphylococcus | FAIL | FAIL | FAIL | PASS | PASS | PASS | PASS |
| aureus | 2.51 | 3.85 | 0 | 5.23 | 5.94 | 5.77 | 5.18 |
| NCTC 8325 | | | | | | | |
| Pseudomonas | PASS | FAIL | FAIL | FAIL | PASS | FAIL | PASS |
| aeruginosa | 5.97 | 0 | 0 | 2.58 | 5.96 | 0 | 5.25 |
| NCIMB 10434 | | | | | | | |
| Candida | FAIL | FAIL | FAIL | PASS | PASS | FAIL | PASS |
| albicans | 1.42 | 2.19 | 0 | 4.9 | 4.9 | 0.29 | 6.25 |
| ATCC 10231 | | | | | | | |
| Aspergillus | FAIL | PASS | FAIL | FAIL | FAIL | PASS | PASS |
| Brasiliensis | 2.32 | 5.67 | 0.2 | 2.1 | 2.4 | 4.43 | 5.88 |
| ATCC 16404 | | | | | | | |

It is clear from Table 1 that the combination of a source of silver ions, a quaternary ammonium salt and an organic acid as claimed in the present invention was effective against all microorganisms tested and yields broad spectrum antimicrobial performance in the modified AATCC-100 test method.

In contrast, silver chloride alone only showed antimicrobial efficacy against the Pseudomonas aeruginosa. Without being bound by theory, it is hypothesized that the ionic silver is being adversely impacted by prolonged exposure to the species within the simulated wound fluid.

Similarly, the antimicrobial efficacy of the benzalkonium chloride is limited to the Aspergillus Brasiliensis species and this, again, is hypothesized to be due to interactions with species within the simulated wound fluid. The benzalkonium chloride appears to have a gap in its spectrum of efficacy with regards to bacteria and yeast when exposed to the specific conditions of the modified AATCC-100 test.

Lactic acid had no antimicrobial efficacy within the modified AATCC-100 test when used alone. Lactic acid is not typically acknowledged as an antimicrobial species in isolation.

Combinations of Silver Chloride and Benzalkonium Chloride (BKC) do yield some improvement in antimicrobial performance in the modified AATCC-100 test method across the two bacteria and the yeast and mould species. However, this combination of ingredients is not sufficient to yield the log 4 reduction required.

Combinations of Silver Chloride and Lactic acid yield a significant improvement in antimicrobial performance in the modified AATCC-100 test method for the bacteria species and the candida albicans yeast, but no improvement in the silver species performance against the Aspergillus Brasiliensis mould.

Surprisingly, it is in the presence of a source of silver ions, a quaternary ammonium salt and an organic acid that a synergistic effect is seen and antimicrobial efficacy across all microorganisms in the modified AATCC-100 test method is observed.

It is of course to be understood that the present invention is not intended to be restricted to the foregoing examples which are described by way of example only.

1. An antimicrobial component for a wound dressing comprising a source of silver ions, a quaternary ammonium salt and an organic acid.

2. An antimicrobial component according to claim 1, wherein the source of silver ions is a silver salt.

3. An antimicrobial component according to claim 2, wherein the silver salt is silver chloride.

4. An antimicrobial component according to claim 1, wherein the quaternary ammonium salt is selected from the group consisting of benzalkonium chloride, benzethonium chloride and cetyltrimethylammonium chloride.

5. An antimicrobial component according to claim 4, wherein the quaternary ammonium salt is benzalkonium chloride.

6. An antimicrobial component according to claim 1, wherein the organic acid is selected from the group consisting of lactic acid, citric acid, acetic acid and malic acid.

7. An antimicrobial component according to claim 6, wherein the organic acid is lactic acid.

8. An antimicrobial component according to claim 1, wherein the source of silver ions is present in an amount of 2%-5% w/w.

9. An antimicrobial component according to claim 1, wherein the source of silver ions is present in an amount to yield 0.95-1.44 mg/cm2 of silver ions.

10. An antimicrobial component according to claim 1, wherein the quaternary ammonium salt is present in an amount of 0.2-0.5% w/w.

11. An antimicrobial component according to claim 1, wherein the quaternary ammonium salt is present in an amount of 0.16-0.24 mg/cm2.

12. An antimicrobial component according to claim 1, wherein the organic acid is present in an amount of 0.5%-1.5% w/w.

13. An antimicrobial component according to claim 1, wherein the organic acid is present in an amount of 0.28-0.34 mg/cm2.

14. An antimicrobial component according to claim 1, wherein the component comprises an absorbent material.

15. An antimicrobial component according to claim 14, wherein the absorbent material is a polyurethane foam.

16. An antimicrobial component according to claim 14, wherein the absorbent material is a non-woven material.

17. An antimicrobial component according to claim 16, wherein the non-woven material is fluff pulp.

18. An antimicrobial component according to claim 14, wherein the absorbent material comprises a gelling fibre.

19. An antimicrobial component according to claim 14, wherein the absorbent material is a polyurethane foam and a non-woven material.

20. An antimicrobial component claim 18, wherein the gelling fibre is selected from the group consisting of carboxymethylcellulose, hydroxyethylcellulose, alginate and chitosan salt.

21. An antimicrobial component according to claim 1, wherein the component has a laminate structure.

22. An antimicrobial component to claim 21, wherein the laminate structure comprises a first layer of polyurethane foam and a second layer of polyurethane foam, wherein the first layer comprises the source of silver ions and the organic acid, and wherein the second layer comprises the quaternary ammonium salt.

23. An antimicrobial component to claim 21, wherein the laminate structure comprises a first layer of polyurethane foam and a second layer of polyurethane foam, and wherein the first layer comprises the source of silver ions, the organic acid and the quaternary ammonium salt.

24. An antimicrobial component to claim 21, wherein the laminate structure comprises a first layer of polyurethane foam and a second layer of non-woven material, and wherein the first layer comprises the source of silver ions, the quaternary ammonium salt and the organic acid.

25. A method of manufacturing an antimicrobial component, comprising the step of bringing together a source of silver ions, a quaternary ammonium salt and an organic acid.

26. A method according to claim 25, comprising incorporating the source of silver ions, the quaternary ammonium salt and the organic acid into an absorbent material.

27. A method according to claim 26, comprising the steps of:
a) incorporating a source of silver ions and an organic acid into a first absorbent material to form a first layer.
b) incorporating a quaternary ammonium salt into a second absorbent material to form a second layer; and
c) bringing together the first layer and the second layer.

28. A wound dressing comprising an antimicrobial component according to claim 1.

29. A method of manufacturing a wound dressing, comprising the steps of:
a) providing a wound contact layer and a backing layer; and
b) locating an antimicrobial component according to claim 1 between the wound contact layer and the backing layer.

30. A method of manufacturing a wound dressing, comprising the steps of:
a) providing a wound contact layer and a backing layer; and
b) locating an antimicrobial component according to claim 1 between the wound contact layer and the backing layer; and
c) welding a first border region of the wound contact layer to a second border region of the backing layer.

31. An antimicrobial component according to claim 1 for use as a therapeutic agent.

32. An antimicrobial component according to any of claim 1 for use in killing or inhibiting the growth of microorganisms.

33. A wound dressing according to claim 28 for use in absorbing fluid discharged from a physiological target, or for use in stemming a flow of a fluid discharged from a physiological target site.
